(19) United States
(12) Patent Application Publication
(10) Pub. No.: US 2025/0083661 A1
(43) Pub. Date: Mar. 13, 2025

(54) SYSTEM AND METHOD TO DETECT AND MITIGATE HAZARDOUS GASES AND AIR QUALITY DEGRADATION NEAR VEHICLES

(71) Applicant: GM Global Technology Operations LLC, Detroit, MI (US)

(72) Inventors: Eric T. Hosey, Rochester Hills, MI (US); Matt Neely, Rochester, MI (US); Matthew Edward Gilbert-Eyres, Rochester Hills, MI (US); Russell A. Patenaude, Macomb Township, MI (US); Adam Lee Wright, Livonia, MI (US)

(73) Assignee: GM Global Technology Operations LLC, Detroit, MI (US)

(21) Appl. No.: 18/465,501

(22) Filed: Sep. 12, 2023

(51) Int. Cl. B60W 30/00; B60H 1/00; B60K 28/00; B60Q 9/00; E05F 15/72

(52) U.S. Cl. CPC B60W 30/00; B60H 1/008; B60H 1/00828; B60H 1/00849; B60K 28/00; B60Q 9/00; E05F 15/72; E05Y 2900/531; E05Y 2900/546; E05Y 2900/55

(57) ABSTRACT

A system for a vehicle includes data processing hardware, memory hardware in communication with the data processing hardware, the memory hardware storing instructions that when executed on the data processing hardware cause the data processing hardware to perform operations including detecting an airborne pollutant within a vehicle cabin, determining whether the airborne pollutant exceeds a pollutant threshold, and reducing the level of airborne pollutant within the vehicle cabin when the airborne pollutant exceeds the pollutant threshold by controlling at least one vehicle system.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against present disclosure.

The present disclosure relates generally to a system and method to detect and mitigate hazardous gases and air quality degradation near vehicles.

Prolonged exposure to hazardous gases, pollutants, and/or poor air quality has the potential to negatively impact human health. Such hazardous gases include carbon monoxide and carbon dioxide, which stem from common household appliances used for heating and cooking in the case of carbon monoxide and from the burning of fossil fuels such as coal, oil, and natural gas in the case of carbon dioxide. Carbon monoxide can lead to immediate poisoning depending on the length and type of exposure while carbon dioxide can cause trouble concentrating, headaches, dizziness, and drowsiness and can eventually lead to poisoning if the exposure to carbon dioxide is prolonged.

Pollutants are found in almost all environments and can be manmade or naturally occurring. For example, manmade pollutants can include volatile organic compounds (VOCs) such as those found in household paints and/or released during off-gassing of plastics. Naturally occurring pollutants occur in nature and can include mold and radon gas, which is a radioactive gas that has no color, smell, or taste. The foregoing pollutants can cause toxicity, headaches, difficulty breathing, and/or certain types of cancer.

As with pollutants, poor air quality can be found in almost all environments and can be the result of human activity, naturally occurring events, or a combination of both. For example, poor air quality can include high or low air pressure, high or low humidity, and/or increased particulate matter within a given volume of air. Air pressure and humidity are naturally occurring and are the result of changes in the weather. Likewise, particulate matter may be the result of naturally occurring events such as wildfires or sandstorms. Particulate matter can also be the result of human activity and can be the result of smog from emissions or soot from fires.

Regardless of the particular threat to human health (i.e., hazardous gases, pollutants, or air quality), monitoring for such threats can lead to early detection which, in turn, can mitigate the harm caused by the threat. In almost all instances, early detection can allow a person to remove the threat, can remove people from a potentially dangerous situation, and/or can provide early assistance to a person experiencing the effects of the threat.

### SUMMARY

A system for a vehicle is provided and includes data processing hardware, memory hardware in communication with the data processing hardware, the memory hardware storing instructions that when executed on the data processing hardware cause the data processing hardware to perform operations including detecting an airborne pollutant within a vehicle cabin, determining whether the airborne pollutant exceeds a pollutant threshold, and reducing the level of airborne pollutant within the vehicle cabin when the airborne pollutant exceeds the pollutant threshold by controlling at least one vehicle system.

The system may include one or more of the following optional features. For example, controlling at least one vehicle system may include instructing an engine control module to turn off an engine of the vehicle. Further, controlling at least one vehicle system may include instructing at least one window motor to move a window from a closed state to an open state. Additionally or alternatively, controlling at least one vehicle system may include instructing a heating, ventilating, and air conditioning (HVAC) system to turn on a fan and/or instructing a body closure mechanism to open a body closure panel of the vehicle.

In one configuration, the level of airborne pollutant within the vehicle cabin is communicated to a user interface device (UID). Additionally or alternatively, the system may be incorporated into a vehicle.

In another configuration, a system for a vehicle is provided and includes data processing hardware, memory hardware in communication with the data processing hardware, the memory hardware storing instructions that when executed on the data processing hardware cause the data processing hardware to perform operations including detecting an airborne pollutant outside of a vehicle cabin, determining whether the airborne pollutant exceeds a pollutant threshold, and restricting entry of the airborne pollutant into the vehicle cabin when the airborne pollutant exceeds the pollutant threshold by controlling at least one vehicle system.

The system may include one or more of the following optional features. For example, controlling at least one vehicle system may include instructing an engine control module to turn off an engine of the vehicle. Further, controlling at least one vehicle system may include instructing at least one window motor to move a window from an open state to a closed state. Additionally or alternatively, controlling at least one vehicle system may include instructing a heating, ventilating, and air conditioning (HVAC) system to turn off and/or instructing a body closure mechanism to close or maintain a body closure panel of the vehicle in a closed state.

In one configuration, the level of airborne pollutant within the vehicle cabin is communicated to a user interface device (UID). Additionally or alternatively, the system may be incorporated into a vehicle.

In yet another configuration, a system for a vehicle is provided and includes data processing hardware, memory hardware in communication with the data processing hardware, the memory hardware storing instructions that when executed on the data processing hardware cause the data processing hardware to perform operations including detecting an airborne pollutant proximate to a vehicle cabin, determining whether the airborne pollutant exceeds a pollutant threshold, and reducing the airborne pollutant when the airborne pollutant exceeds the pollutant threshold by controlling external equipment located a predetermined distance from the vehicle.

The system may include one or more of the following optional features. For example, controlling external equipment may include instructing the external equipment to turn off. Further, the system may instruct at least one window motor to move a window from an open state to a closed state to restrict entry of the airborne pollutant into the vehicle cabin. Additionally or alternatively, the system may instruct a heating, ventilating, and air conditioning (HVAC) system to turn off to restrict entry of the airborne pollutant into the vehicle cabin and/or may instruct a body closure mechanism to close or maintain a body closure panel of the vehicle in a closed state to restrict entry of the airborne pollutant into the vehicle cabin. Finally, the system may be incorporated into a vehicle.

### BRIEF DESCRIPTION OF THE DRAWINGS

The drawings described herein are for illustrative purposes only of selected configurations and are not intended to limit the scope of the present disclosure.

FIG. 1 is a perspective view of a vehicle incorporating a threat detection and mitigation system (TDMS) in accordance with the principles of the present disclosure;

FIG. 2 is a schematic view of the vehicle and TDMS of FIG. 1;

FIG. 3 is a schematic view of the TDMS of FIG. 1; and

FIG. 4 is a flowchart detailing operation of the TDMS of FIG. 1.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

In this application, including the definitions below, the term module may be replaced with the term circuit. The term module may refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, or group) that executes code; memory (shared, dedicated, or group) that stores code executed by a processor; other suitable hardware components that provide the described functionality; or a combination of some or all of the above, such as in a system-on-chip.

The term code, as used above, may include software, firmware, and/or microcode, and may refer to programs, routines, functions, classes, and/or objects. The term shared processor encompasses a single processor that executes some or all code from multiple modules. The term group processor encompasses a processor that, in combination with additional processors, executes some or all code from one or more modules. The term shared memory encompasses a single memory that stores some or all code from multiple modules. The term group memory encompasses a memory that, in combination with additional memories, stores some or all code from one or more modules. The term memory may be a subset of the term computer-readable medium. The term computer-readable medium does not encompass transitory electrical and electromagnetic signals propagating through a medium, and may therefore be considered tangible and non-transitory memory. Non-limiting examples of a non-transitory memory include a tangible computer readable medium including a nonvolatile memory, magnetic storage, and optical storage.

The apparatuses and methods described in this application may be partially or fully implemented by one or more computer programs executed by one or more processors. The computer programs include processor-executable instructions that are stored on at least one non-transitory tangible computer readable medium. The computer programs may also include and/or rely on stored data.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

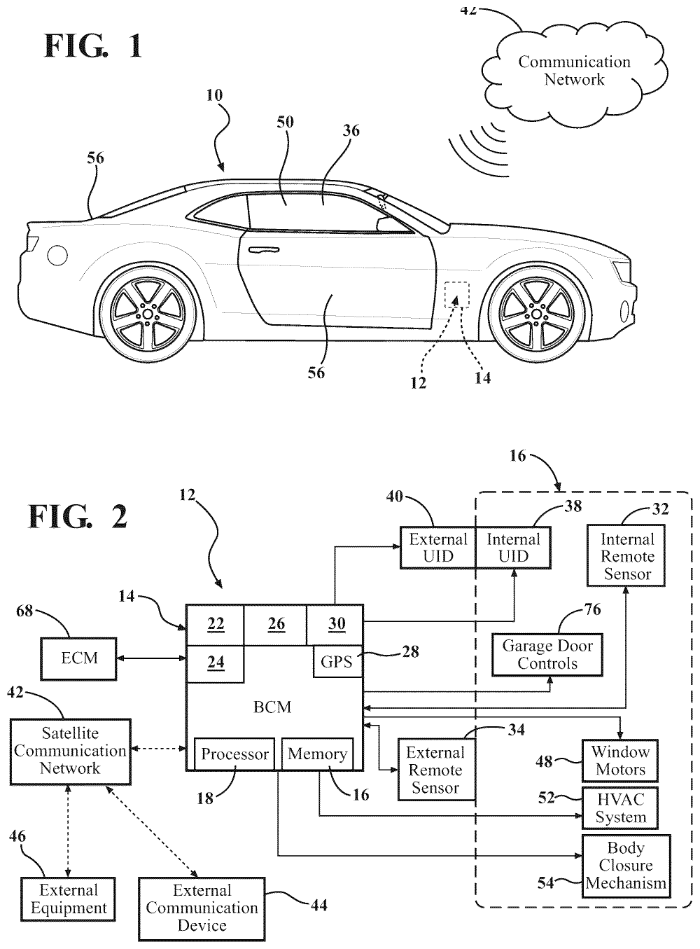

With particular reference to FIGS. 1 and 2, a vehicle 10 is shown in conjunction with a threat detection and mitigation system (TDMS) 12. As will be described in greater detail below, the TDMS 12 may be used to detect and mitigate environmental threats to human health in and around the vehicle 12. For example, the TDMS 12 may be used to detect threats to human health caused by hazardous gases, pollutants, and/or poor air quality and, further, may take steps to mitigate the effects of such threats.

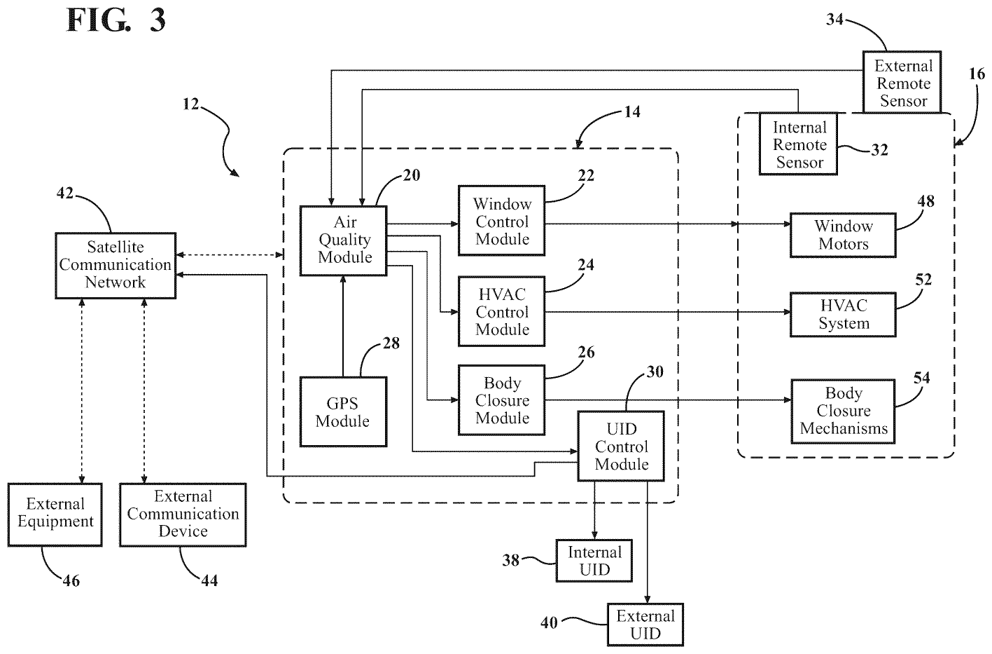

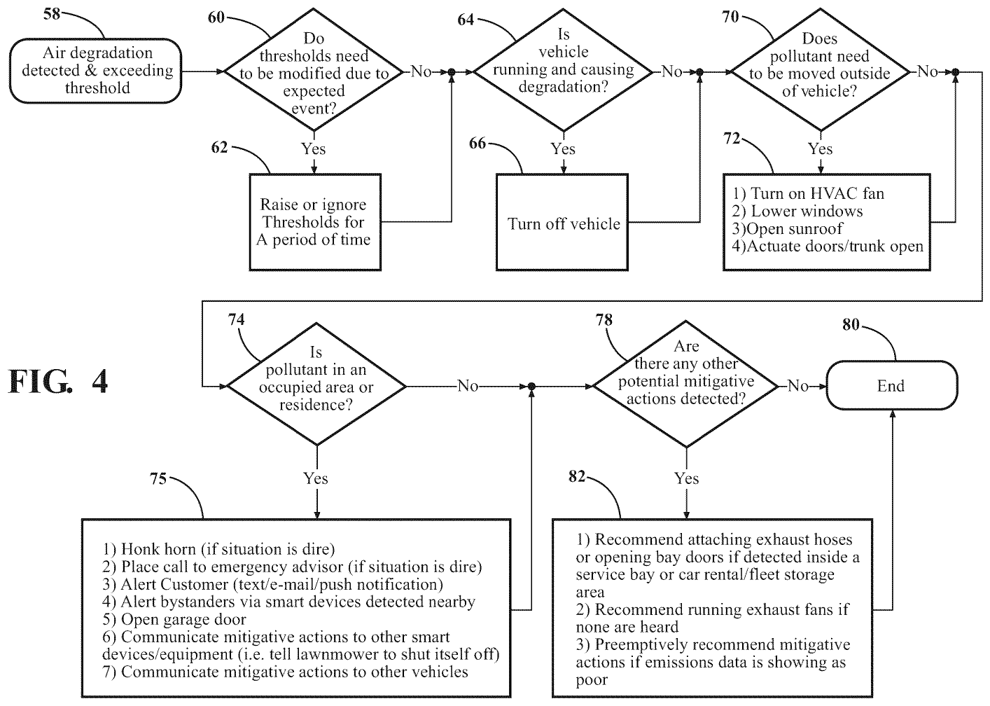

The TDMS 12 may be stored and executed by a body control module (BCM) 14 of the vehicle 10. Specifically, the BCM 14 may store instructions for executing the steps shown in FIG. 4, for example, on memory hardware 16, which may be executed by a processor 18 of the BCM 24. As shown in FIG. 3, the TDMS 12 may include an air quality module 20, a window control module 22, a heating, ventilation, and air conditioning (HVAC) control module 24, a body closure control module 26, a global positioning system (GPS) module 28, and a user interface device (UID) control module 30.

The air quality module 20 may be in communication with the window control module 22, the HVAC control module 24, the body closure control module 26, and the UID control module 30. Additionally, the air quality module 20 may receive information from the GPS module 28 as to the current location of the vehicle 10, as will be described in greater detail below.

The air quality module 20 may also be in communication with an internal remote sensor 32 and an external remote sensor 34. The internal remote sensor 32 may be located within a passenger compartment 36 of the vehicle 10 while the external remote sensor 34 may be located externally from the passenger compartment 36. As such, the internal remote sensor 32 is configured to detect environmental conditions within the passenger compartment 36 while the external sensor 34 is configured to detect environmental conditions outside of the passenger compartment 36 in an area generally surrounding the vehicle 10.

The internal remote sensor 32 and the external remote sensor 34 may each include a series of sensors each in communication with the air quality module 20 of the BCM 14. For example, the internal remote sensor 32 and the external remote sensor 34 may each include a carbon monoxide sensor, a carbon dioxide sensor, an air pressure sensor, a humidity sensor, a particulate sensor configured to detect particulate matter in a volume of air, a radon sensor, a mold sensor, a sensor configured to detect the presence of a volatile organic compound (VOC), and/or a camera configured to capture an image or video of an area within the passenger compartment 36 in the case of the internal remote sensor 32 or an area external from the passenger compartment 36 in the case of the external remote sensor 34.

Information from the internal remote sensor 32 and the external remote sensor 34 may be received and used by the air quality module 20 of the TDMS 12 to monitor conditions within and external from the passenger compartment 36 to detect a threat and, further, to mitigate the threat once detected.

The air quality module 20 may communicate information from one or more of the internal remote sensor 32 and the external remote sensor 34 to the UID control module 30. This information may be communicated to people located within the passenger compartment 36 via an internal UID 38 and may be communicated to people located external from the passenger compartment 38 via an external UID 40. The internal UID 38 may include a display configured to display information received from the air quality module 20 to vehicle occupants. For example, if the internal remote sensor 32 detects an elevated level of carbon dioxide, the air quality module 20 can communicate this information to vehicle occupants via the UID control module 30 and the internal UID 38. This information can also be communicated to people located externally from the vehicle 10 by communicating the information to an external UID 40 such as, for example, a cellular phone or tablet. The information communicated via the internal UID 38 and the external UID 40 may be visually communicated by way of a screen (not shown) associated with the internal UID 38 or the external UID 40. Additionally or alternatively, the information communicated via the internal UID 38 and the external UID 40 may be audibly communicated by way of a speaker (not shown) associated with the internal UID 38 or the external UID 40.

Information from the air quality module 20 may also be transmitted from the BCM 14 to a communication network such as, for example, a satellite communication network 42. The satellite communication network 42 may be in communication with the UID control module 30 to provide information from the air quality module 20 to the internal UID 38 and/or the external UID 40. Additionally, the satellite communication network 42 may be in communication with an external communication device 44 such as a cellular phone or tablet. Finally, the satellite communication network 42 may be in communication with one or more pieces of equipment 46 that may be located proximate to the vehicle 10.

With continued reference to FIG. 3, the window control module 22 is shown in communication with window motors 48 associated with windows 50 of the vehicle 10. The window motors 48 are configured to move the windows 50 between a closed state (FIG. 1) and an open state. The window control module 22 may be in communication with the window motors 48 to selectively move the windows 50 between the closed state and the open state based on information received from the air quality module 20. Specifically, if an airborne threat such as elevated levels of carbon dioxide is detected within the passenger compartment 36 by the internal remote sensor 32, the air quality module 20 may alert the window control module 22. The window control module 22 will then energize one or more of the window motors 48 to move one or more of the windows 50 into the open state to evacuate the threat from within the passenger compartment 36.

The HVAC control module 24 is shown in communication with an HVAC system 52 of the vehicle 10. The HVAC system 52 may include a fan (not shown) configured to circulate air within the passenger compartment 36. Accordingly, in the above example, if an airborne threat is detected within the passenger compartment 36 by the internal remote sensor 32, the HVAC control module 24 may instruct the HVAC system 52 to turn on the fan associated with the HVAC system 52 in an effort to force the threat out of the open windows 50. In this example, the window control module 22 and the HVAC control module 24 work in conjunction with one another to open one or more of the windows 50 and force air from the passenger compartment 38 via the open window(s) in an effort to remove the threat from within the passenger compartment 36.

The body closure control module 26 is shown in communication with one or more body closure mechanism 54 of the vehicle 10. The body closure mechanism 54 may control one or more release mechanisms (none shown) associated with one or more body closure panels 56 of the vehicle 10. For example, the body closure mechanism 54 may be associated with a body closure panel 56 associated with a rear opening of a sport utility vehicle (SUV) or hatchback or may be associated with a body closure panel 56 permitting access to the passenger compartment 36 such as, for example, a passenger door. The body closure mechanism 54 may be configured to move the body closure panel 56 from a closed state to an open state in response to instructions from the body closure module 26.

In the foregoing example, if an airborne threat is detected within the passenger compartment 36 by the internal remote sensor 32, the body closure control module 26 may instruct one or more body closure mechanisms 54 to open one or more body panels 56. In this example, the window control module 22, the HVAC control module 24, and the body closure control module 26 work in conjunction with one another to open one or more of the windows 50, to open one or more of the body closure panels 56, and to force air from the passenger compartment 38 via the open window(s) and body closure panel(s) 56 in an effort to remove the threat from within the passenger compartment 36. It should be appreciated that while the window control module 22, the HVAC control module 24, and the body closure control module 26 are described as working together in the foregoing example, these modules 22, 24, 26 could all work independently from one another depending on the severity and/or type of the threat detected.

With particular reference to FIG. 4, operation of the TDMS 12 will be described in detail. As described above, the air quality module 20 is in communication with the internal remote sensor 32 and the external remote sensor 34 and receives information from the sensors 32, 34 regarding the air quality within the passenger compartment 36 and external to the passenger compartment 38. If one or more of the sensors 32, 34 detects the air quality within the passenger compartment 38 or external to the passenger compartment 36—within a predetermined distance from the vehicle 10—being outside of a predetermined threshold at 58, the TDMS 12 first determines whether the particular threshold(s) needs to be modified at 60. For example, if the TDMS 12 has information from an external source such as a news outlet that the area in which the vehicle 10 is located is experiencing wildfires, the TDMS 12 may raise the stored threshold for particulate matter inside the passenger compartment 36 and external to the passenger compartment 36 for a predetermined time period at 62.

The information from the external source may be conveyed to the TDMS 12 via the satellite communication network 42. For example, the satellite communication network 42 may receive information from an external source such as, for example, a news outlet that wildfires are in the area where the vehicle 10 is currently located and/or is traveling toward. The location of the vehicle 10 may be communicated to the air quality module 20 by the GPS module 28. If the location of the wildfire correlates to the location of the vehicle 10 or is located in an area in which the vehicle 10 is traveling—as indicated by the GPS module 28—the air quality module 20 may raise the threshold(s) for the particular threat(s) detected. In this example, the air quality module 20 may raise the threshold for particulate matter within a volume of air to prevent constant warnings for a known threat. Further, the air quality module 20 may ignore the elevated levels of particulate matter and not take corrective action. Namely, the air quality module 20 may not cause action to be taken by any of the window control module 22, the HVAC control module 24, or the body closure control module 26 in response to the elevated levels of particulate matter detected by one or more of the sensors 32, 34.

It should be noted that even if the TDMS 12 determines that one or more threshold needs to be modified at 60 due to an external event and, further, that the threshold can be raised or modified for a period of time at 62, the TDMS 12 may still instruct the UID control module 30 to inform vehicle occupants and/or persons located remote from the vehicle 10 of the detected elevation in particulate matter. For example, the UID control module 30 may display or play an audio message on one or both of the internal UID 38 or the external UID 40 to communicated the decrease in air quality within the passenger compartment 36 and/or external to the passenger compartment 36 to vehicle occupants or other persons associated with the vehicle (i.e., the vehicle owner, vehicle fleet owner, central dispatch center, etc.).

If the TDMS 12 determines at 60 that the thresholds for the various parameters monitored by the sensors 32, 34 do not need to be modified, the TDMS 12 then determines whether the vehicle 10 is running at 64. If the vehicle 10 is running at 64 and is determined to be the cause of the reduced air quality, the TDMS 12 will turn off the vehicle 10 at 66. Specifically, the TDMS 12 will instruct an engine control module (ECM) 68 in communication with the BCM 14 to turn off the vehicle 10. By way of example, if one of the sensors 32, 34 detects an elevated level of carbon dioxide and, further, that the vehicle 10 is determined to be the cause of the elevated levels of carbon dioxide, the TDMS 12 may instruct the ECM 68 to turn off the vehicle 10 to reduce or eliminate the threat (i.e., eliminate the excess levels of carbon dioxide).

If the vehicle 10 is not running or is not the cause of the reduced air quality at 64, the TDMS 12 then determines whether a pollutant associated with the threat detected by one or more of the sensors 32, 34 needs to be evacuated from the vehicle at 70. If the TDMS 12 determines that a pollutant needs to be evacuated from the vehicle 10, the air quality module 20 of the TDMS 12 may instruct the HVAC control module 24 to turn on the fan associated with the HVAC system 52, may instruct the window control module 22 to open one or more of the windows 50 via the window motors 48, and/or may instruct the body closure control module 26 to open one or more body closure panels 56 via at least one body closure mechanism 54 at 72. For example, if an elevated level of particulate matter is detected within the passenger compartment 36 by the internal remote sensor 32, the air quality module 20 may open a sunroof (not shown) and/or the windows 50 of the vehicle 10 by instructing the window control module 22 to actuate one or more window motors 48.

If the TDMS 12 does not determine that a pollutant needs to be removed from within the passenger compartment 36 at 70, the TDMS 12 then determines whether there is a reduction in air quality within a predetermined distance around the vehicle 10 at 74. For example, if the vehicle 10 is parked at a home or residence, the TDMS 12 may determine at 74 whether there is a reduction in air quality at the home or residence. Finally, regardless of whether the vehicle 10 is at a home or at a residence, when a reduction in air quality is detected by the external remote sensor 34 in an area of the vehicle 10 where people are located, the TDMS 12 may take action to mitigate the reduction in air quality. The TDMS 12 may determine the presence of people around the vehicle 10 by using a camera of at least one of the sensors 32, 34.

If the TDMS 12 determines a pollutant in an area around the vehicle 10 (i.e., in an occupied area or residence) at 74, the TDMS 12 may honk a horn (not shown) of the vehicle 10, may place a call to a police department or fire department via the communication network 42, may alert an owner of the vehicle 10 and/or people living in the area via the external UID 40, the communication network 42, and/or the external communication device 44, or may alert bystanders via the external UID 40, the communication network 42, and/or the external communication device 44 at 75. Additionally, the TDMS 12 may open a garage door (not shown) by sending an open signal to a garage door control 76 (FIG. 2) in an effort to evacuate the threat from an area around the vehicle 10 if the vehicle 10 is located in a garage. In this scenario, the TDMS 12 may know a location of the vehicle 10 based on information from the GPS module 28. If the air quality module 20 determines that an air quality threshold is exceeded in a predetermined area around the vehicle 10 based on information from the external remote sensor 34, the TDMS 12 may instruct the garage door controls 76 to open a garage door to evacuate the air within the garage, thereby mitigating the threat.

In addition to the foregoing, the TDMS 12 may communicate with other external equipment 46 such as a smart lawnmower to turn off the external equipment 46 if one or both of the sensors 32, 34 detects a threat (i.e., reduced air quality) in combination with the vehicle 10 being within a predetermined distance of the external equipment 46. For example, if external equipment 46 is running and one of the sensors 32, 34 detects a decrease in air quality (i.e., an increase in carbon dioxide) such that a threshold is exceeded, the air quality module 20 may instruct the external equipment 46 to turn itself off to reduce the carbon dioxide levels near the vehicle 10. Again, the TDMS 12 will know whether the vehicle 10 is within the predetermined distance relative to the external equipment 46 based on information received from the GPS module 28.

If a pollutant level external to the vehicle 10 is determined to exceed a threshold, the air quality module 20 can take additional action to inhibit the pollutant from entering the passenger compartment 36. For example, the air quality module 20 can instruct the window control module 22 to close all windows 50, can instruct the HVAC control module 24 to turn the HVAC system 52 off or only to circulate air within the passenger compartment 36 (i.e., do not draw external air into the passenger compartment 36), and/or can instruct the body closure control module 26 to keep all body closure panels 56 in the closed state.

In addition to the foregoing, the TDMS 12 may use the communication network 42 to communicate with other vehicles (none shown) in an area proximate to the vehicle 10 to let the other vehicles know of the reduced air quality. For example, if other vehicles are within a predetermined distance of the vehicle 10, the vehicle 10 may communicate with the other vehicles to let the other vehicles know that a pollutant threshold is being exceeded, the nature of the pollutant, and/or the actions taken by the TDMS 12 to mitigate the reduction in air quality. For example, if one or more of the sensors 32, 34 detects an increase in carbon dioxide such that the threshold for carbon dioxide in and/or around the vehicle 10 is exceeded and, further, that the vehicle 10 is not the source of the increased levels of carbon dioxide at 64, the TDMS 12 may communicate with other nearby vehicles to alert the vehicles of the increased levels of carbon dioxide. This way, the other vehicles may take action to mitigate the reduction in air quality.

If the TDMS 12 determines that the pollutant is not in an occupied area or residence at 74, the TDMS 12 determines whether there are any other potential ways to mitigate the reduction in air quality at 78. If not, the TDMS 12 does not take any action at 80. If there are other actions the TDMS 12 can take to mitigate the reduction in air quality at 78, the TDMS 12 moves to 82 and, depending on the pollutant threshold exceeded, the nature of the pollutant, the location of the pollutant, and/or the location of the vehicle 10, the TDMS 12 may improve the air quality in and around the vehicle 10 by taking further steps at 82.

For example, the TDMS 12 may determine that the vehicle 10 is located in a service garage (not shown) based on information from the GPS module 28 and/or a camera associated with one or both of the sensors 32, 34. If one or both of the sensors 32, 34 detects an elevated level of carbon dioxide, this information is sent to the air quality module 20. The air quality module 20 may determine that the increased level of carbon dioxide exceeds the threshold for acceptable levels of carbon dioxide and may recommend taking action to reduce the levels of carbon dioxide around the vehicle 10. For example, the air quality module 20 may communicate to the internal UID 38, the external UID 40, the communication network 42, and/or the external communication device 44 that the threshold for carbon dioxide has been exceeded and, further, that based on the location of the vehicle 10 (i.e., in a service garage), may recommend attaching an exhaust hose to a tailpipe (not shown) of the vehicle 10. Further, the air quality module 20 may additionally or alternatively recommend running an exhaust fan (not shown) in the service garage to evacuate the air within the service garage, thereby removing the threat/pollutant from the garage. Finally, the air quality module 20 may recommend service for the vehicle 10 if emissions data for the vehicle 10 recorded by the module 20 is below acceptable standards.

As described, the TDMS 12 may be used to identify and mitigate threats based on air quality levels in and around a vehicle 10 falling below acceptable standards. For example, the TDMS 12 may communicate to vehicle occupants and/or owners when threshold levels for pollutants such as carbon dioxide are exceeded. Further, the TDMS 12 can take action to improve the air quality by controlling vehicle systems (i.e., turning the vehicle 10 off, opening a window 50, opening a body closure panel 56, and/or turning on a fan of an HVAC system 52) or by controlling external systems such as external equipment 46. Finally, the TDMS 12 can recommend taking actions to remove or reduce the pollutant by alerting vehicle occupants or other people in close proximity to the vehicle to take action (i.e., alerting an occupant or technician to attach an exhaust hose to the tailpipe of the vehicle 10 and/or to turn on an exhaust fan associated with an area in which the vehicle 10 is located).

A number of implementations have been described. Nevertheless, it will be understood that various modifications may be made without departing from the spirit and scope of the disclosure. Accordingly, other implementations are within the scope of the following claims.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

What is claimed is:

1. A system for a vehicle comprising:
data processing hardware;
memory hardware in communication with the data processing hardware, the memory hardware storing instructions that when executed on the data processing hardware cause the data processing hardware to perform operations comprising:
detecting an airborne pollutant within a vehicle cabin;
determining whether the airborne pollutant exceeds a pollutant threshold; and
reducing the level of airborne pollutant within the vehicle cabin when the airborne pollutant exceeds the pollutant threshold by controlling at least one vehicle system.

2. The system of claim 1, wherein controlling at least one vehicle system includes instructing an engine control module to turn off an engine of the vehicle.

3. The system of claim 1, wherein controlling at least one vehicle system includes instructing at least one window motor to move a window from a closed state to an open state.

4. The system of claim 1, wherein controlling at least one vehicle system includes instructing a heating, ventilating, and air conditioning (HVAC) system to turn on a fan.

5. The system of claim 1, wherein controlling at least one vehicle system includes instructing a body closure mechanism to open a body closure panel of the vehicle.

6. The system of claim 1, further comprising communicating the level of airborne pollutant within the vehicle cabin to a user interface device (UID).

7. A vehicle incorporating the system of claim 1.

8. A system for a vehicle comprising:
data processing hardware;
memory hardware in communication with the data processing hardware, the memory hardware storing instructions that when executed on the data processing hardware cause the data processing hardware to perform operations comprising:
detecting an airborne pollutant outside of a vehicle cabin;
determining whether the airborne pollutant exceeds a pollutant threshold; and
restricting entry of the airborne pollutant into the vehicle cabin when the airborne pollutant exceeds the pollutant threshold by controlling at least one vehicle system.

9. The system of claim 8, wherein controlling at least one vehicle system includes instructing an engine control module to turn off an engine of the vehicle.

10. The system of claim 8, wherein controlling at least one vehicle system includes instructing at least one window motor to move a window from an open state to a closed state.

11. The system of claim 8, wherein controlling at least one vehicle system includes instructing a heating, ventilating, and air conditioning (HVAC) system to turn off.

12. The system of claim 8, wherein controlling at least one vehicle system includes instructing a body closure mechanism to close or maintain a body closure panel of the vehicle in a closed state.

13. The system of claim 8, further comprising communicating the level of airborne pollutant outside of the vehicle cabin to a user interface device (UID).

14. A vehicle incorporating the system of claim 8.

15. A system for a vehicle comprising:
data processing hardware;
memory hardware in communication with the data processing hardware, the memory hardware storing instructions that when executed on the data processing hardware cause the data processing hardware to perform operations comprising:
detecting an airborne pollutant proximate to a vehicle cabin, a source of the airborne pollutant being disposed external from or within the vehicle cabin;
determining whether the airborne pollutant exceeds a pollutant threshold, the data processing hardware configured to compare a level of the airborne pollutant with the pollutant threshold to determine if the airborne pollutant exceeds the pollutant threshold; and
reducing the airborne pollutant when the airborne pollutant exceeds the pollutant threshold by controlling external equipment located a predetermined distance from the vehicle, the external equipment being disposed remote from the vehicle cabin or disposed within the vehicle cabin.

16. The system of claim 15, wherein controlling external equipment includes instructing the external equipment to turn off.

17. The system of claim 15, further comprising instructing at least one window motor to move a window from an open state to a closed state to restrict entry of the airborne pollutant into the vehicle cabin.

18. The system of claim 15, further comprising instructing a heating, ventilating, and air conditioning (HVAC) system to turn off to restrict entry of the airborne pollutant into the vehicle cabin.

19. The system of claim 15, further comprising instructing a body closure mechanism to close or maintain a body closure panel of the vehicle in a closed state to restrict entry of the airborne pollutant into the vehicle cabin.

20. A vehicle incorporating the system of claim 15.
